SEQUENCE LISTING (4 sequences)

SEQ ID NO: 1 - DNA, 8561 bp, Homo sapiens
catgtcgggctccacacagcttgtggcacagacgtggagggccactgagccccgctacccgccccacagcctttcctacccagtgcagatcgcccggacgcacacggacgtcgggctcctggagtaccagcaccactcccgcgactatgcctcccacctgtcgccgggctccatcatccagccccagcggcggaggccctccctgctgtctgagttccagcccgggaatgaacggtcccaggagctccacctgcggccagagtcccactcatacctgcccgagctggggaagtcagagatggagttcattgaaagcaagcgccctcggctagagctgctgcctgaccccctgctgcgaccgtcacccctgctggccacgggccagcctgcgggatctgaagacctcaccaaggaccgtagcctgacgggcaagctggaaccggtgtctccccccagccccccgcacactgaccctgagctggagctggtgccgccacggctgtccaaggaggagctgatccagaacatggaccgcgtggaccgagagatcaccatggtagagcagcagatctctaagctgaagaagaagcagcaacagctggaggaggaggctgccaagccgcccgagcctgagaagcccgtgtcaccgccgcccatcgagtcgaagcaccgcagcctggtgcagatcatctacgacgagaaccggaagaaggctgaagctgcacatcggattctggaaggcctggggccccaggtggagctgccgctgtacaaccagccctccgacacccggcagtatcatgagaacatcaaaataaaccaggcgatgcggaagaagctaatcttgtacttcaagaggaggaatcacgctcggaaacaatggaagcagaagttctgccagcgctatgaccagctcatggaggccttggaaaaaaaggtggagcgcatcgaaaacaacccgcgccggcgggccaaggagagcaaggtgcgcgagtactacgaaaagcagttccctgagatccgcaagcagcgcgagctgcaggagcgcatgcagagcagggtgggccagcggggcagtgggctgtccatgtcggccgcccgcagcgagcacgaggtgtcagagatcatcgatggcctctcagagcaggagaacctggagaagcagatgcgccagctggccgtgatcccgcccatgctgtacgacgctgaccagcagcgcatcaagttcatcaacatgaacgggcttatggccgaccccatgaaggtgtacaaagaccgccaggtcatgaacatgtggagtgagcaggagaaggagaccttccgggagaagttcatgcagcatcccaagaactttggcctgatcgcatcattcctggagaggaagacagtggctgagtgcgtcctctattactacctgactaagaagaatgagaactataagagcctggtgagacggagctatcggcgccgcggcaagagccagcagcaacaacagcagcagcagcagcagcagcagcagcagcagcagcagcccatgccccgcagcagccaggaggagaaagatgagaaggagaaggaaaaggaggcggagaaggaggaggagaagccggaggtggagaacgacaaggaagacctcctcaaggagaagacagacgacacctcaggggaggacaacgacgagaaggaggctgtggcctccaaaggccgcaaaactgccaacagccagggaagacgcaaaggccgcatcacccgctcaatggctaatgaggccaacagcgaggaggccatcaccccccagcagagcgccgagctggcctccatggagctgaatgagagttctcgctggacagaagaagaaatggaaacagccaagaaaggtctcctggaacacggccgcaactggtcggccatcgcccggatggtgggctccaagactgtgtcgcagtgtaagaacttctacttcaactacaagaagaggcagaacctcgatgagatcttgcagcagcacaagctgaagatggagaaggagaggaacgcgcggaggaagaagaagaaagcgccggcggcggccagcgaggaggctgcattcccgcccgtggtggaggatgaggagatggaggcgtcgggcgtgagcggaaatgaggaggagatggtggaggaggctgaagccttacatgcctctgggaatgaggtgcccagaggggaatgcagtggcccagccactgtcaacaacagctcagacaccgagagcatcccctctcctcacactgaggccgccaaggacacagggcagaatgggcccaagcccccagccaccctgggcgccgacgggccacccccaggcccacccaccccaccacggaggacatcccgggcccccattgagcccaccccggcctctgaagccaccggagcccctacgcccccaccagcacccccatcgccctctgcacctcctcctgtggtccccaaggaggagaaggaggaggagaccgcagcagcgcccccagtggaggagggggaggagcagaagccccccgcggctgaggagctggcagtggacacagggaaggccgaggagcccgtcaagagcgagtgcacggaggaagccgaggaggggccggccaagggcaaggacgcggaggccgctgaggccacggccgagggggcgctcaaggcagagaagaaggagggcgggagcggcagggccaccactgccaagagctcgggcgccccccaggacagcgactccagtgctacctgcagtgcagacgaggtggatgaggccgagggcggcgacaagaaccggctgctgtccccaaggcccagcctcctcaccccgactggcgacccccgggccaatgcctcaccccagaagccactggacctgaagcagctgaagcagcgagcggctgccatcccccccatccaggtcaccaaagtccatgagcccccccgggaggacgcagctcccaccaagccagctcccccagccccaccgccaccgcaaaacctgcagccggagagcgacgcccctcagcagcctggcagcagcccccggggcaagagcaggagcccggcaccccccgccgacaaggaggccttcgcagccgaggcccagaagctgcctggggaccccccttgctggacttccggcctgcccttccccgtgcccccccgtgaggtgatcaaggcctccccgcatgccccggacccctcagccttctcctacgctccacctggtcacccactgcccctgggcctccatgacactgcccggcccgtcctgccgcgcccacccaccatctccaacccgcctcccctcatctcctctgccaagcaccccagcgtcctcgagaggcaaataggtgccatctcccaaggaatgtcggtccagctccacgtcccgtactcagagcatgccaaggccccggtgggccctgtcaccatggggctgcccctgcccatggaccccaaaaagctggcacccttcagcggagtgaagcaggagcagctgtccccacggggccaggctgggccaccggagagcctgggggtgcccacagcccaggaggcgtccgtgctgagagggacagctctgggctcagttccgggcggaagcatcaccaaaggcattcccagcacacgggtgccctcggacagcgccatcacataccgcggctccatcacccacggcacgccagctgacgtcctgtacaagggcaccatcaccaggatcatcggcgaggacagcccgagtcgcttggaccgcggccgggaggacagcctgcccaagggccacgtcatctacgaaggcaagaagggccacgtcttgtcctatgagggtggcatgtctgtgacccagtgctccaaggaggacggcagaagcagctcaggacccccccatgagacggccgcccccaagcgcacctatgacatgatggagggccgcgtgggcagagccatctcctcagccagcatcgaaggtctcatgggccgtgccatcccgccggagcgacacagcccccaccacctcaaagagcagcaccacatccgcgggtccatcacacaagggatccctcggtcctacgtggaggcacaggaggactacctgcgtcgggaggccaagctcctaaagcgggagggcacgcctccgcccccaccgccctcacgggacctgaccgaggcctacaagacgcaggccctgggccccctgaagctgaagccggcccatgagggcctggtggccacggtgaaggaggcgggccgctccatccatgagatcccgcgcgaggagctgcggcacacgcccgagctgcccctggccccgcggccgctcaaggagggctccatcacgcagggcaccccgctcaagtacgacaccggcgcgtccaccactggctccaaaaagcacgacgtacgctccctcatcggcagccccggccggacgttcccacccgtgcacccgctggatgtgatggccgacgcccgggcactggaacgtgcctgctacgaggagagcctgaagagccggccagggaccgccagcagctcggggggctccattgcgcgcggcgccccggtcattgtgcctgagctgggtaagccgcggcagagccccctgacctatgaggaccacggggcaccctttgccggccacctcccacgaggttcgcccgtgaccatgcgggagcccacgccgcgcctgcaggagggcagcctttcgtccagcaaggcatcccaggaccgaaagctgacgtcgacgcctcgtgagatcgccaagtccccgcacagcaccgtgcccgagcaccacccacaccccatctcgccctatgagcacctgcttcggggcgtgagtggcgtggacctgtatcgcagccacatccccctggccttcgaccccacctccataccccgcggcatccctctggacgcagccgctgcctactacctgccccgacacctggcccccaaccccacctacccgcacctgtacccaccctacctcatccgcggctaccccgacacggcggcgctggagaaccggcagaccatcatcaatgactacatcacctcgcagcagatgcaccacaacacggccaccgccatggcccagcgagctgatatgctgaggggcctctcgccccgcgagtcctcgctggcactcaactacgctgcgggtccccgaggcatcatcgacctgtcccaagtgccacacctgcctgtgctcgtgcccccgacaccaggcaccccagccaccgccatggaccgccttgcctacctccccaccgcgccccagcccttcagcagccgccacagcagctccccactctccccaggaggtccaacacacttgacaaaaccaaccaccacgtcctcgtccgagcgggagcgagaccgggatcgagagcgggaccgggatcgggagcgggaaaagtccatcctcacgtccaccacgacggtggagcacgcacccatctggagacctggtacagagcagagcagcggcagcagcggcagcagcggcgggggtgggggcagcagcagccgccccgcctcccactcccatgcccaccagcactcgcccatctcccctcggacccaggatgccctccagcagagacccagtgtgcttcacaacacaggcatgaagggtatcatcaccgctgtggagcccagcaagcccacggtcctgaggtccacctccacctcctcacccgttcgcccagctgccacattcccacctgccacccactgcccactgggcggcaccctcgatggggtctaccctaccctcatggagcccgtcttgctgcccaaggaggccccccgggtcgcccggccagagcggccccgagcagacaccggccatgccttcctcgccaagcccccagcccgctccgggctggagcccgcctcctcccccagcaagggctcggagccccggcccctagtgcctcctgtctctggccacgccaccatcgcccgcacccctgcgaagaacctcgcacctcaccacgccagcccggacccgccggcgccacctgcctcggcctcggacccgcaccgggaaaagactcaaagtaaacccttttccatccaggaactggaactccgttctctgggttaccacggcagcagctacagccccgaaggggtggagcccgtcagccctgtgagctcacccagtctgacccacgacaaggggctccccaagcacctggaagagctcgacaagagccacctggagggggagctgcggcccaagcagccaggccccgtgaagcttggcggggaggccgcccacctcccacacctgcggccgctgcctgagagccagccctcgtccagcccgctgctccagaccgccccaggggtcaaaggtcaccagcgggtggtcaccctggcccagcacatcagtgaggtcatcacacaggactacacccggcaccacccacagcagctcagcgcacccctgcccgcccccctctactccttccctggggccagctgccccgtcctggacctccgccgcccacccagtgacctctacctcccgcccccggaccatggtgccccggcccgtggctccccccacagcgaagggggcaagaggtctccagagccaaacaagacgtcggtcttgggtggtggtgaggacggtattgaacctgtgtccccaccggagggcatgacggagccagggcactcccggagtgctgtgtacccgctgctgtaccgggatggggaacagacggagcccagcaggatgggctccaagtctccaggcaacaccagccagccgccagccttcttcagcaagctgaccgagagcaactccgccatggtcaagtccaagaagcaagagatcaacaagaagctgaacacccacaaccggaatgagcctgaatacaatatcagccagcctgggacggagatcttcaatatgcccgccatcaccggaacaggccttatgacctatagaagccaggcggtgcaggaacatgccagcaccaacatggggctggaggccataattagaaaggcactcatgggtaaatatgaccagtgggaagagtccccgccgctcagcgccaatgcttttaaccctctgaatgccagtgccagcctgcccgctgctatgcccataaccgctgctgacggacggagtgaccacacactcacctcgccaggtggcggcgggaaggccaaggtctctggcagacccagcagccgaaaagccaagtccccggccccgggcctggcatctggggaccggccaccctctgtctcctcagtgcactcggagggagactgcaaccgccggacgccgctcaccaaccgcgtgtgggaggacaggccctcgtccgcaggttccacgccattcccctacaaccccctgatcatgcggctgcaggcgggtgtcatggcttccccacccccaccgggcctccccgcgggcagcgggcccctcgctggcccccaccacgcctgggacgaggagcccaagccactgctctgctcgcagtacgagacactctccgacagcgagtgactcagaacagggcgggggggggcgggcggtgtcaggtcccagcgagccacaggaacggccctgcaggagcggggcggctgccgactcccccaaccaaggaaggagcccctgagtccgcctgcgcctccatccatctgtccgtccagagccggcatccttgcctgtctaaagccttaactaagactcccgccccgggctggccctgtgcagaccttactcaggggatgtttacctggtgctcgggaagggaggggaaggggccggggagggggcacggcaggcgtgtggcagccacacacaggcggccagggcggccagggacccaaagcaggatgaccacgcacctccacgccactgcctcccccgaatgcatttggaaccaaagtctaaactgagctcgcagcccccgcgccctccctccgcctcccatcccgcttagcgctctggacagatggacgcaggccctgtccagcccccagtgcgctcgttccggtccccacagactgccccagccaacgagattgctggaaaccaagtcaggccaggtgggcggacaaaagggccaggtgcggcctggggggaacggatgctccgaggactggactgtttttttcacacatcgttgccgcagcggtgggaaggaaaggcagatgtaaatgatgtgttggtttacagggtatatttttgataccttcaatgaattaattcagatgttttacgcaaggaaggacttacccagtattactgctgctgtgcttttgatctctgcttaccgttcaagaggcgtgtgcaggccgacagtcggtgaccccatcactcgcaggaccaagggggcggggactgctcgtcacgccccgctgtgtcctccctccctcccttccttgggcagaatgaattcgatgcgtattctgtggccgccatttgcgcagggtggtggtattctgtcatttacacacgtcgttctaattaaaaagcgaattatactccaaaaaaaaaaaaaaaaaaa

SEQ ID NO: 2 - protein, 2517 aa, Homo sapiens
MSGSTQLVAQTWRATEPRYPPHSLSYPVQIARTHTDVGLLEYQHHSRDYASHLSPGSIIQPQRRRPSLLSEFQPGNERSQELHLRPESHSYLPELGKSEMEFIESKRPRLELLPDPLLRPSPLLATGQPAGSEDLTKDRSLTGKLEPVSPPSPPHTDPELELVPPRLSKEELIQNMDRVDREITMVEQQISKLKKKQQQLEEEAAKPPEPEKPVSPPPIESKHRSLVQIIYDENRKKAEAAHRILEGLGPQVELPLYNQPSDTRQYHENIKINQAMRKKLILYFKRRNHARKQWKQKFCQRYDQLMEALEKKVERIENNPRRRAKESKVREYYEKQFPEIRKQRELQERMQSRVGQRGSGLSMSAARSEHEVSEIIDGLSEQENLEKQMRQLAVIPPMLYDADQQRIKFINMNGLMADPMKVYKDRQVMNMWSEQEKETFREKFMQHPKNFGLIASFLERKTVAECVLYYYLTKKNENYKSLVRRSYRRRGKSQQQQQQQQQQQQQQQQQPMPRSSQEEKDEKEKEKEAEKEEEKPEVENDKEDLLKEKTDDTSGEDNDEKEAVASKGRKTANSQGRRKGRITRSMANEANSEEAITPQQSAELASMELNESSRWTEEEMETAKKGLLEHGRNWSAIARMVGSKTVSQCKNFYFNYKKRQNLDEILQQHKLKMEKERNARRKKKKAPAAASEEAAFPPVVEDEEMEASGVSGNEEEMVEEAEALHASGNEVPRGECSGPATVNNSSDTESIPSPHTEAAKDTGQNGPKPPATLGADGPPPGPPTPPRRTSRAPIEPTPASEATGAPTPPPAPPSPSAPPPVVPKEEKEEETAAAPPVEEGEEQKPPAAEELAVDTGKAEEPVKSECTEEAEEGPAKGKDAEAAEATAEGALKAEKKEGGSGRATTAKSSGAPQDSDSSATCSADEVDEAEGGDKNRLLSPRPSLLTPTGDPRANASPQKPLDLKQLKQRAAAIPPIQVTKVHEPPREDAAPTKPAPPAPPPPQNLQPESDAPQQPGSSPRGKSRSPAPPADKEAFAAEAQKLPGDPPCWTSGLPFPVPPREVIKASPHAPDPSAFSYAPPGHPLPLGLHDTARPVLPRPPTISNPPPLISSAKHPSVLERQIGAISQGMSVQLHVPYSEHAKAPVGPVTMGLPLPMDPKKLAPFSGVKQEQLSPRGQAGPPESLGVPTAQEASVLRGTALGSVPGGSITKGIPSTRVPSDSAITYRGSITHGTPADVLYKGTITRIIGEDSPSRLDRGREDSLPKGHVIYEGKKGHVLSYEGGMSVTQCSKEDGRSSSGPPHETAAPKRTYDMMEGRVGRAISSASIEGLMGRAIPPERHSPHHLKEQHHIRGSITQGIPRSYVEAQEDYLRREAKLLKREGTPPPPPPSRDLTEAYKTQALGPLKLKPAHEGLVATVKEAGRSIHEIPREELRHTPELPLAPRPLKEGSITQGTPLKYDTGASTTGSKKHDVRSLIGSPGRTFPPVHPLDVMADARALERACYEESLKSRPGTASSSGGSIARGAPVIVPELGKPRQSPLTYEDHGAPFAGHLPRGSPVTMREPTPRLQEGSLSSSKASQDRKLTSTPREIAKSPHSTVPEHHPHPISPYEHLLRGVSGVDLYRSHIPLAFDPTSIPRGIPLDAAAAYYLPRHLAPNPTYPHLYPPYLIRGYPDTAALENRQTIINDYITSQQMHHNTATAMAQRADMLRGLSPRESSLALNYAAGPRGIIDLSQVPHLPVLVPPTPGTPATAMDRLAYLPTAPQPFSSRHSSSPLSPGGPTHLTKPTTTSSSERERDRDRERDRDREREKSILTSTTTVEHAPIWRPGTEQSSGSSGSSGGGGGSSSRPASHSHAHQHSPISPRTQDALQQRPSVLHNTGMKGIITAVEPSKPTVLRSTSTSSPVRPAATFPPATHCPLGGTLDGVYPTLMEPVLLPKEAPRVARPERPRADTGHAFLAKPPARSGLEPASSPSKGSEPRPLVPPVSGHATIARTPAKNLAPHHASPDPPAPPASASDPHREKTQSKPFSIQELELRSLGYHGSSYSPEGVEPVSPVSSPSLTHDKGLPKHLEELDKSHLEGELRPKQPGPVKLGGEAAHLPHLRPLPESQPSSSPLLQTAPGVKGHQRVVTLAQHISEVITQDYTRHHPQQLSAPLPAPLYSFPGASCPVLDLRRPPSDLYLPPPDHGAPARGSPHSEGGKRSPEPNKTSVLGGGEDGIEPVSPPEGMTEPGHSRSAVYPLLYRDGEQTEPSRMGSKSPGNTSQPPAFFSKLTESNSAMVKSKKQEINKKLNTHNRNEPEYNISQPGTEIFNMPAITGTGLMTYRSQAVQEHASTNMGLEAIIRKALMGKYDQWEESPPLSANAFNPLNASASLPAAMPITAADGRSDHTLTSPGGGGKAKVSGRPSSRKAKSPAPGLASGDRPPSVSSVHSEGDCNRRTPLTNRVWEDRPSSAGSTPFPYNPLIMRLQAGVMASPPPPGLPAGSGPLAGPHHAWDEEPKPLLCSQYETLSDSE

SEQ ID NO: 3 - DNA, 22 bp, Homo sapiens
acagtggctgagtgcgtcctct

SEQ ID NO: 4 - DNA, 22 bp, Homo sapiens
acgtggagctggaccgacattc

(19) United States
(12) Patent Application Publication
(10) Pub. No.: US 2008/0070254 A1
(43) Pub. Date: Mar. 20, 2008

(54) Method of prognosis of metastasis by detection of FRA12E fragile site within the SMRT gene/locus at chromosome 12q24

(76) Inventor: Lionel J. Coignet, East Amherst, NY (US)

Correspondence Address: HODGSON RUSS LLP;THE GUARANTY BUILDING, 140 PEARL STREET, SUITE 100, BUFFALO, NY, 14202-4040, US

(21) Appl. No.: 11/827,566

(22) Filed: Jul. 12, 2007

Related U.S. Application Data
(63) Continuation-in-part of application No. 11/052,344, filed on Feb. 7, 2005.
(60) Provisional application No. 60/542,538, filed on Feb. 6, 2004.
(60) Provisional application No. 60/830,364, filed on Jul. 12, 2006.

(51) Int. Cl. C12Q 1/68; C12Q 1/02

(52) U.S. Cl. 435/6; 435/29

(57) ABSTRACT

Provided are methods for prognostic prediction of metastasis. The method comprises identifying the presence or absence of a fragile site FRA12E in 12q24 at the SMRT gene locus. The presence of FRA12E in an individual can be determined in cell samples obtained from an individual by hybridization techniques such as fluorescence in situ hybridization techniques or by analyzing alterations of expression of the SMRT gene. The presence of this FRA12E is indicative of higher likelihood of metastasis than if this fragile site is absent.

This application claims priority to U.S. provisional patent application Ser. No. 60/830,364, filed on Jul. 12, 2006, and is a continuation-in-part of U.S. patent application Ser. No. 11/052,344, filed on Feb. 7, 2005, which claims priority to U.S. provisional application Ser. No. 60/542,538 filed on Feb. 6, 2004, the disclosures of each of which are incorporated herein by reference.

### FIELD OF THE INVENTION

This invention relates generally to the field of cancer and more particularly to tools and methods for prognosis of metastatic disease.

### DESCRIPTION OF RELATED ART

Metastasis is the spread of cancer from a primary site and the formation of new tumors in distant organs. When cancer is detected at an early stage, before it has spread, it can often be treated successfully by surgery or local irradiation. However, when cancer is detected after it has metastasized, treatments are much less successful. Furthermore, for many patients in whom there is no evidence of metastasis at the time of their initial diagnosis, metastases can occur at a later time, even decades after apparently successful primary treatment.

Metastases can show an organ-specific pattern of spread. Breast cancer (BC), the most frequent cancer in the female population of industrialized countries, often metastasizes to bone. Metastases to bone occur in >70% of patients with advance disease. Despite some advances in chemotherapeutic regimens, it is virtually impossible to cure breast cancer-induced metastasis and osteolysis. Metastasis of other cancers is also organ specific, such as prostate cancer, which is known to spread to bone.

A persistent clinical challenge that spans all types of cancer has been to predict, among a group of individuals having the same types of cancer and with similar demographics, risk factors, and disease characteristics, which patients will actually progress from localized to metastatic disease and which will remain disease-free following initial therapy. Despite the clinical importance of metastasis, much remains to be learned about the biology of the metastatic process as well as prediction and predisposition markers. In part, knowledge is limited because metastasis is a ‘hidden’ process and diagnosis is typically made after metastasis has already occurred. Many molecular factors have been identified as contributing to the formation of detectable metastases. However, the identification of molecules and genes that are associated with a metastatic end point does not, in itself, provide information about how these molecules contribute to the metastatic process and how the process is driven. This knowledge will be important for providing the biological context in which to apply the rapidly increasing information about molecular contributors to metastasis.

To develop metastatic lesions, tumor cells must be able to accomplish each step in the multistep process while avoiding host immune surveillance (1). A series of cellular events appears associated with all metastatic processes. These include interactions of the cancer cells with the surrounding stromal cells; interactions with the extracellular matrix leading to matrix recognition, cell-attachment, release of bioactive matrix-bound factors and matrix destruction for tumor expansion; formation of tumor vasculature or angiogenesis; and escape from immunoprotection and from cell death (2).

A similar pattern of events takes place in the transformation of non-Hodgkin's lymphomas (NHLs) where the development of the full neoplastic phenotype most likely depends on the acquisition of multiple genetic events, including the concurrent activation of synergistic dominant oncogenes and loss of tumor suppressor gene functions (3,4).

While the mechanism of malignancy or metastasis are not completely understood, genetic breakage is one mechanism by which functional loss of tumor suppressor gene activity may occur. Chromosomal locations in which genetic breakage may be induced are known as fragile sites. Fragile site breakages may be induced, for example by a chemical (such as aphidicolin), by hypoxia, or by physical force (such as a physical shock to the media containing the DNA). Fragile sites have been shown to be involved in some malignancies in which the fragile site lies within known genes, such as the FHIT gene (chromosome 3p) in lung cancer, and where small deletions are consistently observed on chromosome 3 (17,18). These fragile sites are inherited in a dominant Mendelian fashion. They are also known to contain specific motifs repeated more than 200 times. It has previously been shown that two fragile sites exist on the long arm of chromosome 12. FRA12B is located at 12q24.13 (19) and FRA12E has been located at 12q24.2-3 (20). It has also been previously estimated that approximately 5% of the human population is positive for one of these two fragile sites. However, it is not known whether there is a relationship of these fragile sites with cancer or with metastasis. Accordingly, there is an ongoing need to identify and develop novel markers, and in particular for markers useful for predicting a risk of metastases.

### SUMMARY OF THE INVENTION

The present invention provides a method for the prognostic prediction of metastasis in a broad range of cancers. The method comprises detecting the presence or absence of a fragile site in the SMRT gene locus in human chromosome 12. The presence of this fragile site at this locus is indicative of higher likelihood of metastasis than if the fragile site is absent. The presence of this fragile site may be detected directly by using as probes nucleotide sequences which can hybridize to the SMRT locus in the 12q24 region or indirectly by detecting altered expression of the SMRT gene. Examples of nucleotide sequences that can be used as probes include vectors comprising SMRT locus specific polynucleotides, such as SMRT-specific BAC clones, and SMRT locus specific oligonucleotides.

In one embodiment, the method for detecting the presence of FRA12E site on chromosome 12 in the 12q24 region of the SMRT locus comprises the steps of contacting a genomic DNA sample from an individual with one or more labeled BAC clones which comprise sequences of the 12q24 region at the SMRT gene locus and determining specific binding of the probes. In a preferred embodiment, the specific binding of the probes is determined by fluorescent in situ hybridization (FISH).

In another embodiment, primers complementary to FRA12E specific sequences in the SMRT locus may be used to amplify FRA12E specific sequences, when such sequences are present, and thereafter detect the amplified products to ascertain the presence of FRA12E.

In another aspect of the invention, since the fragile site FRA12E is located within or in close proximity to the SMRT gene, a prognostic evaluation predictive of metastasis can also be carried out by determining the expression of the SMRT protein by immunodetection such as by ELISA, Western blotting, fluorescence labeling and the like. Furthermore, the presence of the FRA12E site, and therefore an increased likelihood of metastasis, can also be determined by analyzing expression of the SMRT gene and detecting alterations in SMRT mRNA or SMRT protein. In alternative embodiments, the method can be performed by detecting, in situ or otherwise, alterations in the form or mix of metabolic byproducts that also indicate alterations in SMRT gene expression due to the presence of the FRA12E site.

### BRIEF DESCRIPTION OF THE DRAWINGS

This patent or application contains at least one drawing executed in color. Copies of this patent or patent application publication with color drawings will be provided by the Patent Office upon request and payment of the necessary fee.

FIGS. 1A and 1B provide photographic results of Western blot analysis of different malignant cell lines with an anti-SMRT antibody. FIG. 1A: CTV1=AML cell line (control), all the other lines are transformed NHLs. FIG. 1B: MCF7: metastatic breast cancer cell line and MCF10A, its non malignant counterpart; Col2: metastatic colorectal cancer; LnCAP: non-metastatic prostate; PC3: metastatic prostate; Hec1a: metastatic endometrial cancer; A549: metastatic non-small cell lung carcinoma; PIG1: immortalized keratinocytes; F002: melanoma in situ; M1123, M14 and G24: metastatic melanoma cell lines.

FIGS. 2A and 2B provide photographic representations of immunostaining with an anti-SMRT antibody on breast cancer paired samples: FIG. 2A) primary tumor and FIG. 2B) metastatic tumor from the same patient. Nine paired samples were studied. Seven out of nine showed the same differential pattern of expression as shown in this figure i.e., positive in the primary tumor and negative in the metastatic tumor. The two other cases showed positive staining for both primary and metastatic tumor. The difference between the two groups of patients (+ or − SMRT in the metastatic tumor) is that average time from primary diagnosis to metastatic disease diagnosis goes from 2 years (for the +/−cases) to 9 years (for the +/+cases). FIGS. 2C and 2D are representations of immunostaining with anti-SMRT antibody for benign prostate tumor (2C) showing positive staining and metastatic tumor (2D) showing a lack of staining.

FIGS. 3A and 3B provide a schematic representation mapping of the RPCI11 BAC clones used to detect and map the FRA12E fragile site within the SMRT gene. FIG. 3A: BAC clones 339, 665 and 667 encompassing the SMRT locus; BAC 469 can be used as a control probe. FIG. 3B: Schematic representation of the potential spreading of breakages over the SMRT locus.

FIG. 4 is a photographic representation of FISH results illustrating mapping of the FRA12E fragile site within the SMRT gene. Chromosomes were harvested after induction of the fragile sites with aphidicolin and hybridized with the SMRT-specific RPCI11 BAC clones (FIG. 3) (green) and control BAC clone (red). One chromosome 12 shows a normal pattern of hybridization wherein the predominant signal is yellow (due to the overlapping/juxtaposition of the green and the red signals). The other signal (indicated by short arrow) shows a split green signal, next to a main yellow signal indicating that the BAC clones span a DNA double strand break due to activation of the fragile site. The SMRT specific probe hybridized on both sites of the breakage, giving rise to the split FISH signal.

FIG. 5 provides a schematic representation of the detection of the FRA12E fragile site in peripheral blood samples from normal controls and patients with breast cancer, with or without metastatic disease. The graph provides the percentage of metaphase chromosomes with disrupted FISH signals (indicative of FRA12E breaks) in a patient. Each dot represents a patient sample, the dotted line, the cut off value that allows the best discrimination between the different groups and determining the status of a sample (FRA12E carrier or not). The two last categories represent additional patient samples tested that have metastatic prostate cancer and melanoma.

FIG. 6 provides a schematic representation of data from detection of the FRA12E fragile site in peripheral blood samples from normal controls and patients with prostate cancer, with or without metastatic disease. The graph provides the percentage of metaphase chromosomes with disrupted FISH signals (indicative of FRA12E breaks) in a patient. Each dot represents a patient sample, the dotted line, the cut off value that allows the best discrimination between the different groups and determining the status of a sample (FRA12E carrier or not).

FIGS. 7A and 7B provide photographic and graphical representations, respectively, of an RT-PCR time-course analysis of SMRT mRNA subsequent to incubation of 5256 FS+ (with the FRA12E site) and EBV Lin FS− (without the FRA12E site) cell lines with aphidicolin for the various times shown.

FIG. 8 is a photographic representation of electrophoretic separation of RT-PCR products and demonstrates a decrease in SMRT mRNA expression in NHL cell lines as compared with a control cell line (CTV1).

FIG. 9 is a photographic representation of mmunofluorescence results performed by incubating the indicated cell lines with aphidicolin and detecting SMRT protein expression using a commercially available rabbit polyclonal anti-SMRT antibody (Affinity BioRreagents) followed by a secondary anti-rabbit-FITC. The counterstain is DAPI.

### DETAILED DESCRIPTION OF THE INVENTION

The present invention provides a method for prognosis of metastasis by determining whether an individual carries the fragile site FRA12E. The present method is based on the finding that the fragile site FRA12E is located on the human chromosome 12q24 region at the SMRT gene locus. Further, our results in breast cancer, prostate cancer, melanoma, and kidney cancer, and the involvement of SMRT down-regulation in several other cancers, indicate that this specific fragile site localized within the SMRT locus is directly related to a greater risk of damage to the integrity of SMRT, and can ultimately lead to tumor transformation/metastasis.

According to standard fragile site nomenclature, FRA12E is the 5^{th} potential fragile site on human chromosome 12 counting from the telomere of the p arm towards the q telomere. When it is present, it is located at 12q24.2-3 (20), which is also the site of the SMRT gene locus.

SMRT was originally described in 1995 (5) as a protein whose association with nuclear receptors both in solution and bound to DNA response elements is destabilized by ligand. Studies by several groups (6-10) demonstrated that SMRT and a related co-repressor N-CoR (nuclear receptor co-repressor) recruit a transcriptional repression complex, which contains sin3A/B protein and histone deacetylases (HDAC1/2)(11-14) to nuclear receptors. The ability of the “silencing complex” to deacetylate histones results in a condensed chromatin state which can inhibit transcription (15,16). The sequence of the SMRT gene is provided as SEQ ID NO:1, where the open reading frame starts at nucleotide 2 and ends at nucleotide 7551. The SMRT protein amino acid sequence is provided as SEQ ID NO:2.

The method of the present invention makes use observations described herein to provide a method for prognostic determination of metastasis. The method comprises the steps of determining the likelihood of cancer metastasis in an individual comprising detecting in a biological sample from the individual the presence or absence of a FRA12E fragile site in human chromosome 12 at the SMRT gene locus.

In one aspect of the invention, the presence of FRA12E in human chromosome 12 at the SMRT gene locus can be detected in a variety of ways. For example, it could be detected either directly on indirectly. Direct detection could be performed, for example, by detecting the genomic sequence of FRA12E by using PCR, QPCR, in situ PCR, primed in situ hybridization, or Southern Hybridization. It could also be performed by hybridization or in situ hybridization using one or more probes. It could also be performed by amplifying the SMRT gene locus using one of a variety of methods known in the art (such as PCR), then assaying the amplified DNA to determine the presence or absence of FRA12E, for example, by determining the average lengths of the polynucleotides before or after inducing FRA12E to break.

Indirect detection could be performed, for example, by detecting the presence or absence of mRNA for SMRT, preferably after breakage at the FRA12E site. This could be achieved, for example, using semi-quantitative RT-PCR or quantitative RT-PCR (QRTPCR). It could also be performed by northern blot or by using RNA probes for hybridization, for example by in situ hybridization. The presence of FRA12E in human chromosome 12 at the SMRT gene locus could also be indirectly detected by evaluating the levels of the SMRT protein in a sample, for example by immunodetection or immunoassay (such as Western Blot, ELISA, florescent labeling, radioimmunoassay, secretion assay, or immunostaining), nuclear staining (for example with visual or flow cytometry detection), or other non-immunoassays known to those skilled in the art.

The presence of FRA12E in human chromosome 12 at the SMRT gene locus could also be indirectly detected by assaying the levels of metabolic byproducts which result when there is alteration to SMRT expression as a result of the presence of FRA12E. For example, differences in cell secretions due to altered biochemical pathways could be detected by a tissue, blood, or urine test. Alternatively, alterations of cell surface expressed proteins could be detected, for example by antibody labeling and flow cytometry.

It will be apparent to one skilled in the art that this invention encompasses virtually every method by which the presence of FRA12E in human chromosome 12 at the SMRT gene locus could be detected.

In another aspect of the invention, the presence of FRA12E in human chromosome 12 at the SMRT gene locus can be detected in a variety of different biological samples. For example, it could be detected from a sample taken as part of a tumor biopsy. However, since FRA12E is an inherited trait, its presence could be detected in a sample taken from any other part of the body. For example, it could be detected from a blood sample of the patient, from lymphocytes taken from the patient, or from an endothelial or other tissue sample taken from the patient using any of the methods which are known in the art for taking tissue or other biological samples from a patient. The sample could also be taken from a culture of tissue which has been grown from a biological sample taken from a patient. For example, the culture could be an in vitro culture or an in vivo propagation of the tissue either in the patient or in another animal. The culture could also comprise immortalized cells derived from a tissue taken from the patient. Thus, it would be apparent to one skilled in the art that this invention encompasses virtually every method by which a genetic sample might be acquired and/or amplified from a patient.

In certain embodiments, the presence of the fragile site is determined by detecting experimentally induced breakages at the FRA12E fragile site.

In one aspect of the invention is provided a method for detecting the presence of FRA12E site on chromosome 12 in 12q24 at the SMRT locus comprising the steps of contacting a genomic DNA sample from an individual with one or more probes. The probes comprise polynucleotides or oligonucleotides which include sequences complementary to the 12q24 region at the SMRT gene locus; and determining specific binding of the probes.

The sequence of the oligonucleotide for hybridization or PCR amplification will depend upon several factors known in the art. Primarily the sequence of the oligonucleotide will be determined by its capacity to bind to the FRA12E site. This determination is well within the purview of those skilled in the art. Accordingly, one skilled in the art could readily design, for example, a probe of sufficient complementary to SMRT mRNA to hybridize to SMRT mRNA based upon conventional and established nucleic acid hybridization parameters.

Polynucleotides or oligonucleotides (referred to as “probes” herein) that specifically hybridize to the FRA12E site can be provided alone, in phages, plasmids, phagemids, cosmids, yeast artificial chromosomes (YACs), bacterial artificial chromosomes (BACs) or any other suitable vector without limitation. It is well known in the art that probes for nucleic acid hybridization based detections of complimentary sequences can be as small as 20 bases. Thus, for example when using BAC clones as probes, either the entire BAC clone can be used or portions thereof (20 bases or more) such that detection of the fragile site can be effected. For FISH assays an example of a convenient length of probes which are made up of portions of BAC clones is about 1 kilo base. For primers used in amplification reactions, an example of a convenient length is approximately 15 to 25 nucleotides. For instance, one example of a set of primers for performing RT-PCR amplification of SMRT mRNA is SMRT: forward primer: 5′-ACA GTG GCT GAG TGC GTC CTC T-3′ (SEQ ID NO:3); reverse primer: 5′-ACG TGG AGC TGG ACC GAC ATT C-3′ (SEQ ID NO:4).

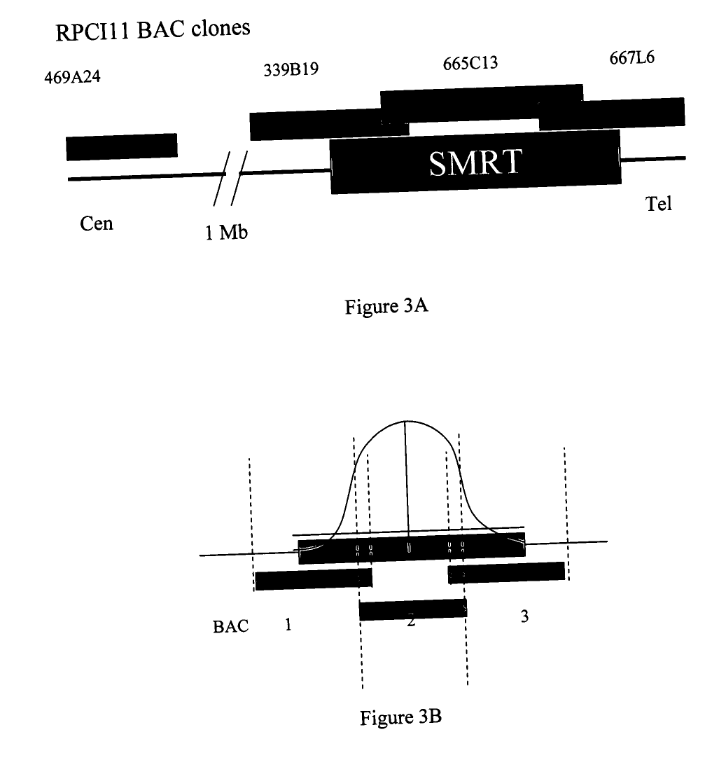

In one embodiment, olignonucleotides can be designed to selectively bind to sequences in the SMRT gene locus which are SMRT gene coding sequences or SMRT gene non-coding sequences. For example, by comparing the sequence of probes which collectively or individually encompass the SMRT gene locus to the SMRT cDNA sequence provided as SEQ ID NO:1, or another cDNA sequence encoding the amino acid sequence of SEQ ID NO:2, wherein such cDNA sequences can be readily recognized by those skilled in the art based on the redundancy of the genetic code, the sequence of SMRT coding regions and SMRT non-coding regions can be ascertained. One example of a combination of probes which collectively encompass the sequence of the SMRT gene is BAC constructs BAC-RP11-339B19, BAC-RP11-665C13, and BAC-RP11-677L6 (see FIG. 3A).

In one embodiment, oligonucleotide probes may be provided as PCR primers for PCR amplification of FRA12E sequences in the non-coding regions of the SMRT locus. Without intending to be bound by any particular theory, it is believed that such FRA12E sequences comprise repeated sequence motifs which contribute to the instability of this fragile site.

In another embodiment, the probes are used in Southern hybridization assays to detect the presence of the FRA12E site. The hybridizations can be carried out on a test sample and compared to a Southern hybridization by standard methods performed on a control sample. Such reactions can be carried out using a single probe or a panel of overlapping probes that span the 12q24 region. Control probes which can hybridize to a region of chromosome 12 outside of the 12q24 region may also be used.

In another embodiment, in situ hybridization techniques can be used. Since a fragile site is a genetic trait, it is present in all cells, therefore, any tissue can be used. For in situ hybridization techniques, typically, cells are harvested from a biological sample using standard techniques. For example, cells can be harvested by centrifuging a biological sample such as blood, and resuspending the pelleted cells. Typically, the cells are resuspended in phosphate-buffered saline (PBS). Alternatively, the cells obtained from blood can be set in culture for from one to a few days (such as 3 days) with phytohemeagglutinin (PHA) to stimulate proliferation of lymphocytes. Prior to harvesting the cells (such as 24 hours prior to harvesting), treatment can be carried out with a DNA polymerase inhibitor (such as aphidicolin) or any other chemical which induces fragile site breakage and therefore facilitates visualization of fragile sites as chromosomal breakage at the FRA12E fragile site.

For visualization of a breakage in the fragile site, after harvesting and treatment with aphidicolin or any other chemical known to induce fragile site breakage, the cells can be fixed, for example, in acid alcohol solutions, acid acetone solutions, or aldehydes such as formaldehyde, paraformaldehyde, and glutaraldehyde. To obtain chromosome preparations, cells are cultured for 1 to 3 days. A blocker (such as Colcemid) is added to the cultures to block the cells in metaphase, where chromosomes are highly condensed and can be visualized. Chromosome preparations are then fixed (such as with a solution of methanol/acetic acid (3:1 vol/vol) and then spread onto slides. Pretreatments (such as RNase and pepsin) can be applied to the preparations to potentially lower the background and to facilitate the penetration of the probes in the nuclei, respectively.

The cell suspension is applied to slides such that the cells are preferably present as a single layer. Cell density can be measured by a light or phase contrast microscope. Prior to in situ hybridization, chromosomal probes and chromosomal DNA are denatured. Denaturation typically is performed by incubating in the presence of high pH, heat (e.g., temperatures from about 70° C. to about 95° C.), organic solvents such as formamide and tetraalkylammonium halides, or combinations thereof. For example, chromosomal DNA can be denatured by a combination of temperatures above 70° C. (e.g., about 73° C.) and a denaturation buffer containing 70% formamide and 2×SSC (0.3M sodium chloride and 0.03 M sodium citrate). Denaturation conditions typically are established such that cell morphology is preserved. Chromosomal probes can be denatured by heat. For example, probes can be heated to about 73° C. for about five minutes.

After denaturation, hybridization can be carried out. This hybridization can be performed using a variety of laboratory techniques known to persons skilled in the art, for example, in situ hybridization, florescent in situ hybridization (FISH), Southern Hybridization, liquid hybridization, or micro arrays (for example using re-sequencing arrays). In various embodiments, hybridization conditions may vary, depending on the concentrations, base compositions, complexities, and lengths of the probes, as well as salt concentrations, temperatures, and length of incubation. The higher the concentration of probe, the higher the probability of forming a hybrid. For example, in situ hybridizations are typically performed in hybridization buffer containing 1-2×SSC, 50% formamide and blocking DNA to suppress non-specific hybridization. In general, hybridization conditions, as described above, include temperatures of about 25° C. to about 55° C., and incubation lengths of about 0.5 hours to about 96 hours. More particularly, hybridization can be performed at about 32° C. to about 40° C. for about 2 to about 16 hours.

Non-specific binding of chromosomal probes to DNA outside of the FRA12E region can be reduced by a series of washes. Temperature and concentration of salt in each wash depend on the desired stringency. For example, for high stringency conditions, washes can be carried out at about 65° C. to about 80° C., using 0.2× to about 2×SSC, and about 0.1% to about 1% of a nonionic detergent such as Nonidet P-40 (NP40). Stringency can be lowered by decreasing the temperature of the washes or by increasing the concentration of salt in the washes.

Chromosomal probes typically are chosen for maximal sensitivity and specificity. The probes generally range from about 50 to about 1×10⁵ nucleotides in length. Chromosomal probes typically are directly labeled with a fluorophore, an organic molecule that fluoresces after absorbing light of lower wavelength/higher energy, but could also be labeled radioactively, chromogenically, or with a tag (such as biotin or digoxygenin) which requires a secondary molecule for detection. In a preferred embodiment, the fluorophore allows the probe to be visualized without a secondary detection molecule. After covalently attaching a fluorophore to a nucleotide, the nucleotide can be directly incorporated into the probe with standard techniques such as nick translation, random priming, and PCR labeling. Alternatively, deoxycytidine nucleotides within the probe can be transaminated with a linker. The fluorophore then is covalently attached to the transaminated deoxycytidine nucleotides. Fluorophores of different colors are chosen such that each chromosomal probe in the set can be distinctly visualized. Suitable fluorophores include: 7-amino-4-methylcoumarin-3-acetic acid (AMCA), Texas Red™ (Molecular Probes, Inc., Eugene, Oreg.), 5-(and-6)-carboxy-X-rhodamine, lissamine rhodamine B, 5-(and-6)-carboxyfluorescein, fluorescein-5-isothiocyanate (FITC), 7-diethylaminocoumarin-3-carboxylic acid, tetramethylrhodamine-5-(and-6)-isothiocyanate, 5-(and-6)-carboxytetramethylrhodamine, 7-hydroxycoumarin-3-carboxylic acid, 6-[fluorescein 5-(and-6)-carboxamido]hexanoic acid, N-(4,4-difluoro-5,7-dimethyl-4-bora-3a,4a diaza-3-indacenepropionic acid, eosin-5-isothiocyanate, erythrosin-5-isothiocyanate, and Cascade™ blue acetylazide (Molecular Probes, Inc., Eugene, Oreg.). Probes are viewed with a fluorescence microscope and an appropriate filter for each fluorophore.

Probes also can be indirectly labeled with, for example, biotin or digoxygenin, or labeled with radioactive isotopes such as ³²P and ³H, although secondary detection molecules or further processing then is required to visualize the probes. For example, a probe indirectly labeled with biotin can be detected by avidin conjugated to a detectable marker. For example, avidin can be conjugated to an enzymatic marker such as alkaline phosphatase or horseradish peroxidase. Enzymatic markers can be detected in standard colorimetric reactions using a substrate and/or a catalyst for the enzyme. Catalysts for alkaline phosphatase include 5-bromo-4-chloro-3-indolylphosphate and nitro blue tetrazolium. Diaminobenzoate can be used as a catalyst for horseradish peroxidase.

Examples of test probes useful for detecting the presence of FRA12E include the entire SMRT gene locus or portions of the SMRT gene locus of suitable complementary and length for specific hybridization to the SMRT gene locus. These sequences can be present as part of BAC clones (such as commercially available clones from the RPCI BAC library). The sequences of these clones are easily accessed if necessary, for example, through GenBank at the National Center for Biotechnology Information (NCBI). Examples include polynucleotides comprising sequences corresponding to genomic BAC constructs BAC-RP11-339B19, BAC-RP11-665C13, BAC-RP11-677L6, BAC-RP11-408118 and BAC-RP11-30G17 (all of which are available to the public from, for example, the Roswell Park Cancer Institute, Buffalo, N.Y.). While any one of these probes or portions thereof as described herein can be used, it is preferable to use more than one probe such that a majority of the SMRT gene locus is encompassed.

In one embodiment, three overlapping probes which span the entire SMRT gene locus are used. For example, by using a panel of probes such as BAC-RP11-339B19, BAC-RP11-665C13 and BAC-RP11-677L6, any breakage of chromosome 12 in the sequence of the entire SMRT gene locus can be assessed. Alternatively, two probes which bind to sequences located at the 3′ and 5′ ends of the SMRT gene respectively, may be employed.

In one embodiment, two BAC clones can be used. For example, we have determined that RP11-677L6 and RP11-665C13 alone encompass a sufficient amount of the SMRT locus such that they can be employed in a FISH assay to determine whether a chromosomal breakage has occurred within the SMRT locus. In this regard, five blood cell samples from five different patients with or without metastatic breast cancer were analyzed by FISH using RP11-677L6 and RP11-665C13. This method successfully identified chromosomal breakage at the SMRT locus to within 5% accuracy of using all three probes.

Thus, BAC clone nucleotide sequences can be used to detect the presence of the FRA12E fragile site as a marker for a likelihood of the progression of cancer to metastatic disease/transformation and as a predictor factor for cancer patients to develop metastatic disease. Negative control probes comprising nucleotide sequences corresponding to regions of chromosome 12 outside the SMRT locus, such as BAC RP11-469A24 (RPCI BAC library), can be used, for example in FISH assays, to increase the accuracy of results.

As an alternative to visualization of chromosomal breakage of the fragile site using in situ visualization techniques such as FISH, alterations in SMRT gene expression can be analyzed in a biological sample obtained from an individual to determine the presence of the FRA12E fragile site, and to therefore determine whether the individual has an increased likelihood of metastasis. For example, a biological sample comprising nucleated cells from an individual can be obtained and treated with aphidicolin or any other agent known to induce fragile site breakage, after which alterations in the expression of SMRT gene expression can be analyzed. In particular, after treatment of the biological sample with such an agent, a reduction in SMRT gene expression as evidenced by a reduction SMRT mRNA and/or SMRT protein expression is indicative that the individual is carrier of the FRA12E fragile site and therefore has an increased risk of metastasis. In this regard, it is demonstrated herein that when cells that contain the FRA12E fragile site are analyzed in this manner, a transient reduction in SMRT mRNA expression occurs between approximately 2 and 5 hours after treating cells with an agent known to induce fragile site breakage. In contrast, cells which do not contain the FRA12E fragile site do not exhibit such transient reduction in SMRT mRNA expression. Therefore, this transient reduction in SMRT mRNA expression is indicative of the presence of the FRA12E fragile site, and therefore also indicative of an increased likelihood of metastasis. It is also demonstrated that when cells that contain the FRA12E fragile site are analyzed, a reduction in SMRT protein expression occurs approximately 18 hours after treating cells with an agent known to induce fragile site breakage. In contrast, cells that do not contain the FRA12E fragile site do not exhibit a reduction in SMRT protein expression. Accordingly, a reduction in SMRT protein expression is indicative of the presence of the FRA12E fragile site, as well as the concomitant increased likelihood of metastasis.

The prognostic test can be used to screen individuals diagnosed with malignancies to predict a likelihood of metastasis. For example, such malignancies may include but are not limited to solid tumors (such as prostate, breast, colorectal, lung (small cell and non-small cell), ovarian, melanoma, urinary system, uterine, endometrial, pancreatic, oral cavity, thyroid, stomach, brain and other nervous system, liver, and esophagial) or hematological cancers (such as Hodgkins Lymphoma, Non-Hodgkins Lymphoma (NHL), chronic and other leukemias, and myeloma). The method could also be used to screen individuals who have been diagnosed with non-malignant neoplasias or other benign or in situ growths which might not be considered “malignant” but which have a certain probability of becoming malignant or metastasizing. This test can also be used in the general population as an identifier of individuals as being carriers of the FRA12E fragile site and whom, if they were to develop cancer, would be at higher risk of metastasis. In this regard, knowing that an individual has a higher risk of metastasis could lead to a more vigilant cancer-detection regimen. In connection with this, we have determined that FRA12E is more prevalent in the cancer population than it is in the general population. Specifically, we found that 5/42 (12%) women without cancer were carriers of the fragile site. By contrast, 20/65 (31%) patients with no evidence of disease after 5 years from their primary diagnostic were carriers of the fragile site and 35/37 (95%) of the metastatic patients were carriers, showing that the cancer population exhibits significant differences in FRA12E status as compared to the general population.

The present method could also be used to ascertain the FRA12E status for individuals who are at higher risk of cancer, such as individuals with a family history of cancer, with genetic markers indicating a predisposition or increased risk for cancer, with exposure to potentially carcinogenic environmental factors (such as carcinogenic chemicals or radiation), or with a history of cancer risk behaviors.

The method of the invention could be used in a variety of applications. Thus, it is another aspect of this invention that the prognostic test could be used to alter the treatment protocol for patients with cancer or could change the follow-up protocols of patients who have been treated for cancer or who have higher risk of getting cancer or of having metastases if they get cancer. Thus in one embodiment, this invention comprises a method of treating cancer comprising administering a test for the presence of FRA12E in the SMRT gene locus then determining a treatment protocol based wholly or in part on the results of that test. For example, we have determined that if an individual is not a FRA12E carrier, the individual has less than a 1% of risk of metastasis. Therefore, such an individual could, for example, be prescribed surgical resection, but forgo chemotherapy. However, individuals who are FRA12E carriers have greater than 50% risk of metastasis. Therefore, they could, for example, be treated earlier with chemotherapy, and/or be treated with higher doses of chemotherapy. In another embodiment, the invention comprises a method of following-up on patients comprising administering a test for the presence of FRA12E in the SMRT gene locus then selecting a follow-up protocol based in part on the results of that test.

In another aspect, kits for performing the prognostic tests provided by the invention are provided. For example the kits could comprise test probes and optionally negative control probes. For example, the kit may comprises probes comprising sequences of BAC clones which hybridize to the 12q24 region of chromosome 12 at the SMRT gene locus. In one embodiment, the test probes are polynucleotides comprising sequences of BAC-RP11-339B19, BAC-RP11-665C13, and BAC-RP11-677L6, or the entire BAC clones. The test probes could have fluorescent labels thereon (such as fluorescein or rhodamine and the like). The negative control probes could have a fluorescent label which is different from the label on the test probes. The kits may also comprise polynucleotide sequences comprising a sequence of sufficient complementary to SMRT mRNA to hybridize to SMRT mRNA. Optionally, the kits could also include a DNA polymerase inhibitor (such as aphidicolin)—either in a solution form or as a powder, or other chemical which could be used to facilitate the visualization of fragile sites. Further, cell lines that have been tested and identified to be either positive or negative for the fragile site could also be included in the kits.

The invention is further described by the examples presented below. These examples are illustrative and not intended to be restrictive in any way.

### EXAMPLE 1

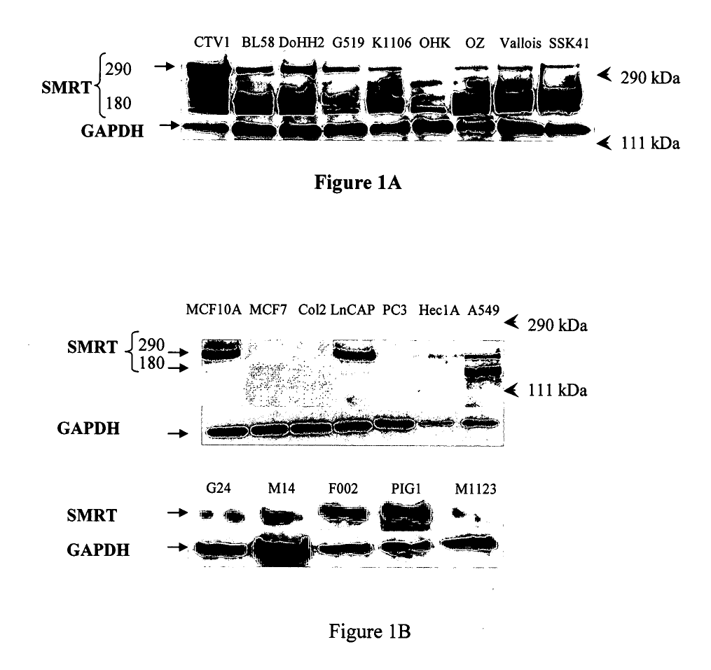

This example demonstrates that the SMRT gene is down-regulated in metastatic cells. To illustrate this, the expression of SMRT gene was determined in several metastatic cell lines. Total protein was extracted from the different cell lines and subjected to Western blotting using a polyclonal anti-SMRT antibody. As shown in FIG. 1, western blot analysis of a series of metastatic cell lines showed a marked SMRT down-regulation of SMRT in most of the cell lines examined.

### EXAMPLE 2

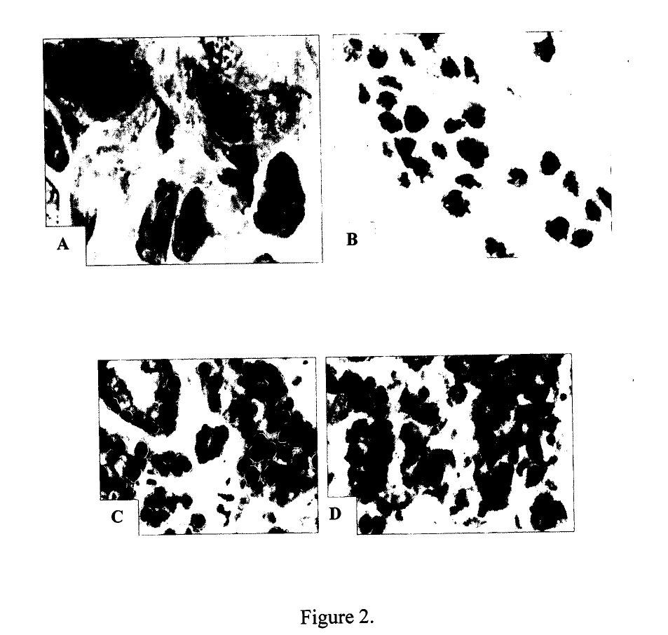

In this example, the down regulation of the SMRT gene was tested in breast cancer (BC). SMRT gene expression in several breast cancer cell lines was determined as described in Example 1. The results demonstrate that BC cell lines as well as samples from BC patient with metastatic disease have altered SMRT expression (FIG. 1B). Further, 7 out of nine paired breast cancer samples (primary and metastatic tumors from the same patient) presented a positive staining in the primary tumor and negative in the metastatic sample. An example is shown in FIG. 2. Similar results were obtained in prostate tumors wherein 6 out of 8 tumors presented a negative staining in the metastatic sample.

### EXAMPLE 3

In this example, immunostaining data was obtained for SMRT from tissue microarrays for samples of breast, prostate, colorectal, lung and ovarian cancer. Each array was made up of 150 to 200 samples. The results are shown in Table 1. Numbers given are percentages of tumors with loss of SMRT expression.

TABLE 1

| | | | | Androgen | Lymph | |
|---|---|---|---|---|---|---|
| | Be- | Pri- | Androgen | Indepen- | Node | Distant |
| Tissue | nign | mary | Dependent | dent | Mets | Mets |
| Prostate | 0 | 10 | 24 | 40 | NA | 67 |
| Breast | NA | 25 | NA | NA | 40 | 77 |
| Colorectal | NA | 10 | NA | NA | 25 | 70 |
| Ovarian | NA | 25 | NA | NA | 40 | 75 |
| Lung | NA | 15 | NA | NA | 25 | 65 |

These data indicate a correlation of the lack of SMRT expression with the stage of the disease, with primary tumors showing less incidence of SMRT negativity.

### EXAMPLE 4

The FRA12E region corresponds to the site of SMRT (12q24.2). We tested the hypothesis that inactivation of SMRT in metastatic/transformed tumors is due to a chromosomal breakage in the SMRT locus due to the presence of this fragile site. Further, if the presence of this fragile site explains the recurrence of breakpoints at 12q24 within the SMRT locus, their presence may represent a predisposition status for these individuals to transformation of NHL or metastasis of solid tumors.

To assess whether the FRA12E fragile site is localized within the SMRT gene, we conducted a study where lymphocytes obtained from healthy individuals were cultured in the presence of aphidicolin (an inducer of fragile sites) and metaphase chromosomes were subsequently prepared as described below. These preparations were then subjected to fluorescence in situ hybridization using a set of SMRT-specific RPCI BAC clones (FIG. 3A).

Three RPCI11 BAC clones encompassing the SMRT locus were used in our experiments and are depicted as green bars in FIG. 3A. These clones have been sequenced through the Human Genome project. The GenBank references for these sequences are: cen: AC068837 (Jul. 13, 2000 entry; BAC-RP11-339B19), AC027706 (May 2, 2000 entry; BAC-RP11-665C13), AC025685 (May 26, 2000 entry; BAC-RP11-677L6) tel. (NCBI website—http://www.ncbi/nlm.nih.gov/). Other BAC clones can correspond to these sequences, such as AC073916 (Mar. 27, 2003 entry; BAC-RP11-408118) and AC069261 (Mar. 27, 2003 entry; BAC-RP111-30G17). Any polynucleotide sequence or a set of polynucleotides or clones which covers part or all of the SMRT locus can be used. In one embodiment, sequences which cover the entire SMRT gene locus are used. This locus has been described in Jiang et al. [21]. Clones lacking this sequence such as a BAC clone telomeric to this group of BACs, can be used as a control probe. An example of a control probe is GenBank reference AC048340 (Apr. 3, 2003 entry; BAC-RP11-469A24).

BAC DNA labeling was done as follows. The BAC DNAs were extracted using a DNA extraction kit (Qiagen). DNA was then subject to nick translation labeling with fluorochrome-conjugated nucleotides using a commercially available kit (Vysis, Downers Grove, Ill.). The SMRT-specific clones were labeled in green whereas the control was labeled in red.

For chromosome preparation, the following procedure was used. One tube of peripheral blood was obtained from cancer patients and controls. The blood was set for a 3-days culture in 10 ml of RPMI medium, supplemented by 10% of fetal calf serum and antibiotics, with 135 μg/ml of PHA. Twenty-four hours before harvesting chromosomes, aphidicolin (0.2 μg/ml) was added to the culture in order to induce fragile sites. Chromosome harvesting was then carried out according to classical cytogenetic techniques for chromosome preparations. Microscope slides were then prepared for hybridization with the labeled BAC clones.

Fluorescence in situ hybridization was carried out as follows. Hybridization was carried out according to established protocols (21). A mixture containing one hundred nanogram of each labeled BAC DNA was applied to the chromosome preparation after denaturation. Hybridization was carried out overnight and slides were washed the next morning. Slides were mounted with antifade solution prior to be observed under a UV microscope with the appropriate filters for the assessment of the signals.

As shown in FIG. 3B, overlapping clones that encompass the entire SMRT locus may be used. Thus, it is preferable to use overlapping clones that encompass a majority of the SMRT locus when performing an analysis using a technique such as FISH.

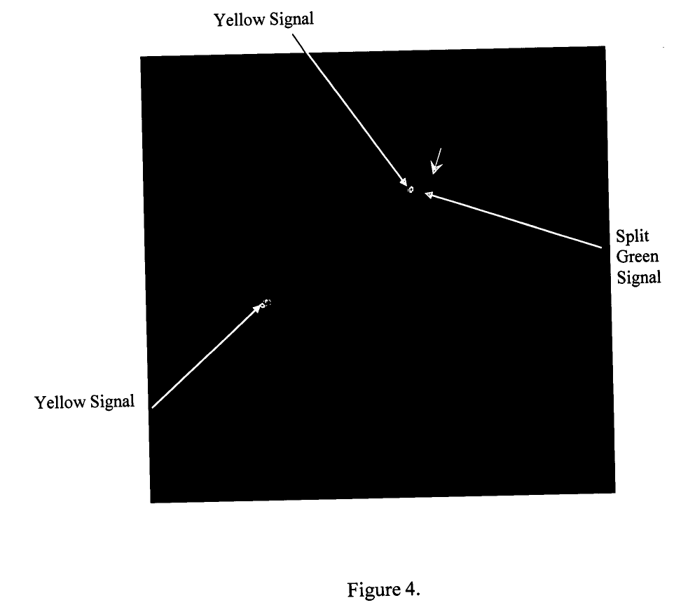

Using this approach, we were able to demonstrate that the FRA12E fragile site is localized within the SMRT gene (FIG. 4). This figure shows two signals. The upper right hand corner signal (identified by the short arrow) is characterized by a large signal in yellow due to the SMRT-specific green signal mixed with the control probe red signal. In addition, split green signals can be observed just to the right of this first yellow signal. The split greet signals represent the presence of the fragile site. The FISH signal present at the bottom left hand corner shows mostly yellow color signal, due to the overlapping/juxtaposition (co-localization) of the SMRT-specific probes green signals with the red signal from the control probe. No split green signal is observed in this case indicating the absence of the fragile site. Furthermore, we have demonstrated that the combination of RP11-677L6 and RP11-665C13 alone encompass a sufficient amount of the SMRT locus to detect breakage of the FRA12E site. Specifically, five samples from five different patients with metastatic disease were analyzed using RP11-677L6 and RP11-665C13 in a FISH assay as set forth in this Example, which successfully identified chromosomal breakage at the SMRT locus to within 5% accuracy of using all three of RP11-339B19, RP11-665C13 and RP11-677L6.

### EXAMPLE 5

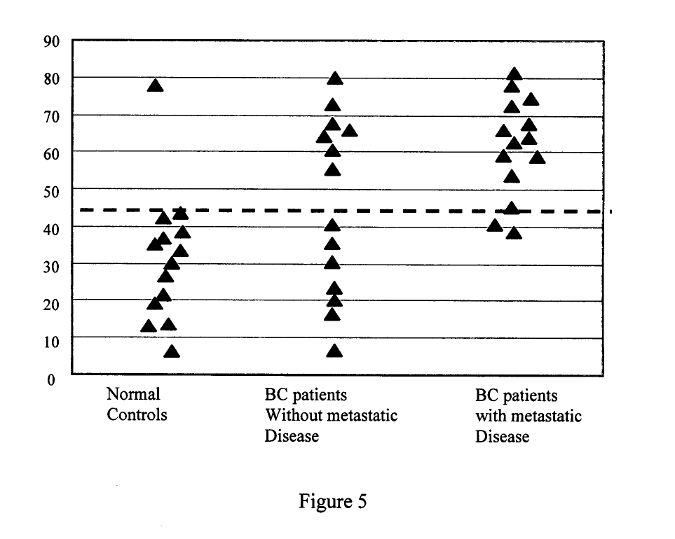

To test if the SMRT alterations observed in transformation/metastasis might be the consequence of the presence of this fragile site (FRA12E) in its sequence, we conducted a study with breast cancer patients, where we compared the incidence of the fragile site in the blood cells of patients with BC and metastatic disease, patients with BC without metastatic disease and normal population. We recruited three groups of patients, with breast cancer with metastatic disease, with breast cancer without metastatic disease and without breast cancer. Peripheral blood was collected and subject to chromosome preparation, as described above. The results obtained in this study (FIG. 5) show that 100% of BC patients presenting “early” metastatic disease are carriers of the fragile site. As indicated in the figure, two metastatic patients showed to be non-carrier. They correspond to one patient with metastatic disease that developed nine years after their primary diagnostic and one case of untreated cancer as compared with 7% in normal controls. In patients with BC without metastatic disease, two groups are represented, 50% non-carrier and 50% carrier. The patients in the non-metastatic group were within 1-2 years of their primary diagnosis. These cases can be followed clinically to see whether those that are carrier of the FRA12E further develop metastatic tumors. Statistical analysis showed that the variation between the mean from the control group and the metastatic group is significant (p=0.000441×10⁻³). Similarly, the variation between the mean from the carrier versus non-carrier in the non-metastatic group is also significant (p=0.00168×10⁻⁶). On the other hand, this analysis shows that the variation between the mean from the control group and the non-carrier from the non-metastatic group as well as the variation between the mean from the metastatic group and the carrier group from the non-metastatic group are not significant (p=0.06 and 0.748, respectively).

As shown in FIG. 5, we tested additionally 4 samples from metastatic prostate cancer (2) and metastatic melanoma (2). Our analysis showed that these patients were also carriers for the FRA12E fragile site

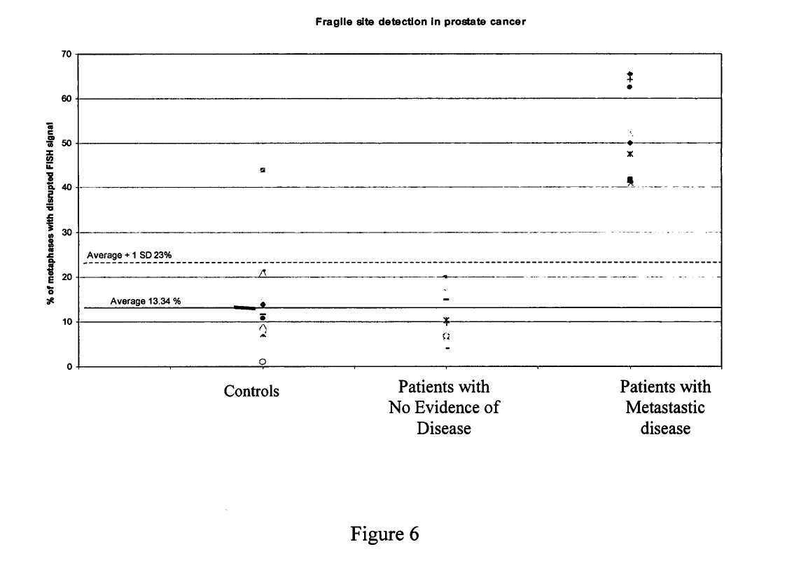

In another illustration of this embodiment, we performed a similar study with prostate cancer patients and controls as shown in FIG. 6. Each group had 14 patients. We similarly found that one control out of 14 was carrier of the fragile site, none of the prostate cancer patients that did not develop metastatic tumors were carrier whereas all of the patients that developed metastatic disease were carriers (p<0.0001). We also tested three samples from patients with metastatic kidney disease and all were shown to be carriers of the FRA12E fragile site.

Therefore, the foregoing data demonstrate that the present invention can be used for prediction of prognosis of metastatic disease from a broad range of cancer types by determining the presence of the FRA12E fragile site.

### EXAMPLE 6

This Example demonstrates methods for determining the presence of the FRA12E fragile site, and thereby whether an individual has an increased risk of metastasis, by analysis of alterations in SMRT gene expression.

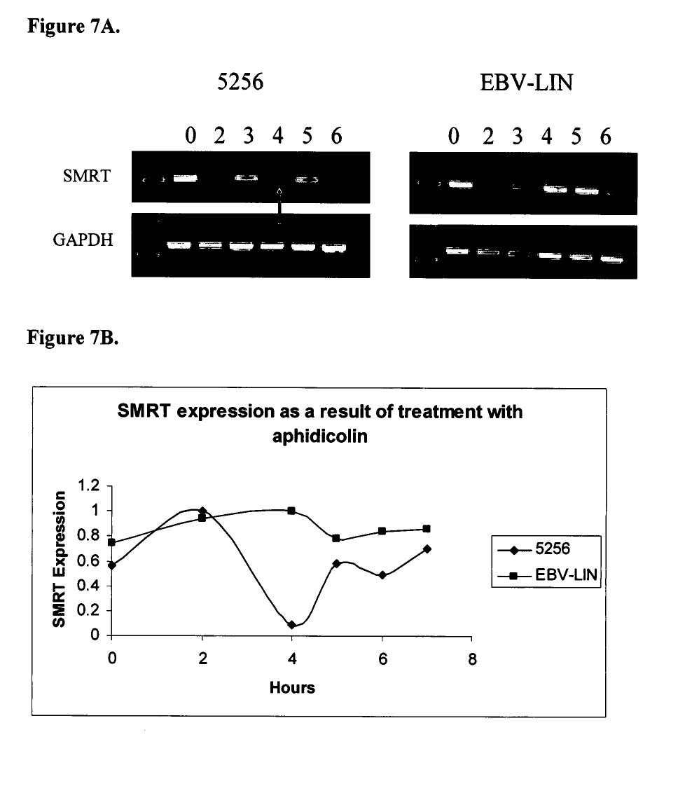

As shown in FIGS. 7A and 7B, RT-PCR time course analysis of SMRT mRNA expression from 5256 cells, which are lymphcotyes immortalized by EBV and are carriers of the FRA12E site, and the control cell line EBV-LIN, which does not contain the FRA12E site, demonstrates that at three hours post-addition of aphidicolin, a transient reduction in SMRT mRNA expression occurs. In particular, the reduction appears approximately during the fourth hour post-addition of aphidicolin, after which SMRT mRNA expression returns to a level similar to that prior to addition of aphidicolin. This transient reduction in SMRT mRNA expression is consistent with the known presence of the FRA12E site in the 5526 cells, whereas the lack of reduction of SMRT mRNA expression between hours 3 and 5 post-addition of aphidicolin in the EBV-LIN cells is consistent with the lack of the FRA12E site in the EBV-LIN cells.

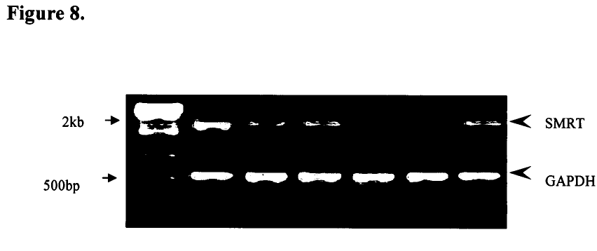

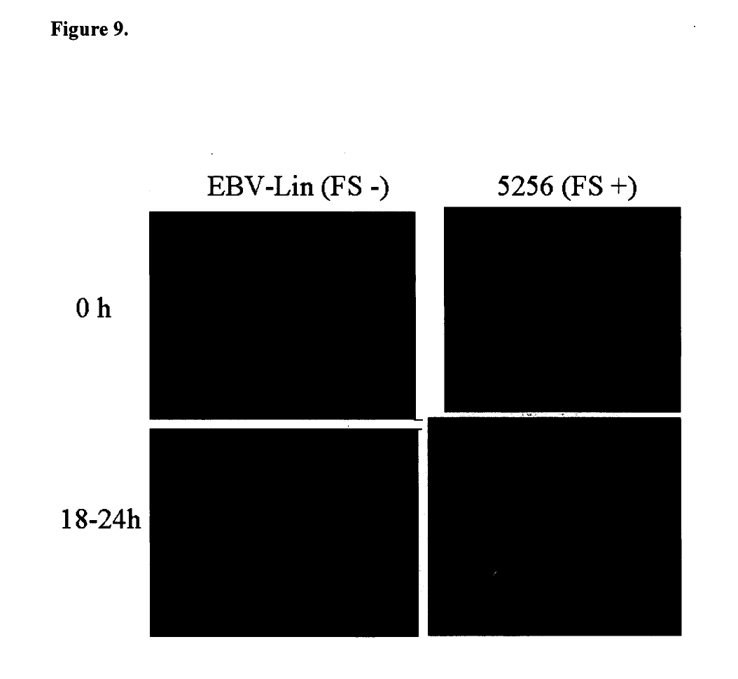

We have also observed that that NHL cells which are known to have a breakage of the FRA12E site have reduced SMRT mRNA expression relative to CTV1 cells, which do not have the disruption, as shown in FIG. 8. Further, as shown in FIG. 9, addition of aphidicolin to cells harboring the FRA12E site (5256 cells) results in a reduction in SMRT protein expression after approximately 18 hours, but addition of aphidicolin to cells which do not contain the FRA12E site (EBV-LIN) does not cause a reduction in SMRT protein expression. Additionally, and as described for Example 1, we have also observed that NHL cells which are known to have a disruption of the FRA12E site have reduced SMRT protein expression (FIG. 1A). Furthermore, there is a lack of SMRT protein expression in the MCF7 metastatic breast cancer cell line, while SMRT protein is expressed in MCF10A, its non malignant counterpart. SMRT protein is also expressed in LnCAP, a non-metastatic prostate cancer, but is absent in PC3, a metastatic prostate cell line. Hec1a is metastatic endometrial cancer line and lacks SMRT protein. Likewise, A549 is a metastatic non-small cell lung carcinoma line which lacks SMRT protein expression, while Col2 is a metastatic colorectal cancer line and also displays a lack of SMRT protein expression (FIG. 1B).

Thus, this Example demonstrates that a reduction in SMRT gene expression is consistent with a metastatic cellular phenotype and a breakage at the FRA12E site in the SMRT locus. This Example also demonstrates that a reduction in SMRT gene expression after adding an agent known to induce breakage of fragile sites to cells can be used to ascertain the presence or absence of the FRA12E site within the cells. Therefore, the method of the invention can facilitate a determination of whether an individual has an increased likelihood of metastasis by ascertaining the FRA12E status of the individual, including by way of analysis of alterations in SMRT gene expression.

While the present invention has been described using the above examples, routine modifications to this invention will be apparent to those skilled in the art and are intended to be within the scope of the invention.

### REFERENCES

1—Yoneda T., Williams P., Hiraga T., Niewolna M., Nishimura R. A bone-seeking clone exhibits different biological properties from the MDA-MB-231 parental breast cancer cells and a brain-seeking clone in vivo and in vitro. J. Bone Miner. Res., 16: 1486-1495, 2001.
2—Fidler I. Modulation of the organ microenvironment for treatment of cancer metastasis. J. Natl. Cancer Inst. (Bethesda), 87: 1588-1592, 1995.
3—Korsmeyer S J (1992). BCL-2 initiates a new category of oncogenes: regulators of cell death. Blood, 80: 879-886.
4—Hahn W C, Counter C M, Lundberg A S, Beijersbergen R L, Brooks M W, Weinberg R A Creation of human tumour cells with defined genetic elements. Nature 1999 Jul. 29; 400(6743):464-8
5—Chen J D, Evans R M A transcriptional co-repressor that interacts with nuclear hormone receptors. Nature 1995 Oct. 5; 377(6548):454-7
6—Heinzel T, Lavinsky R M, Mullen T M, Soderstrom M, Laherty C D, Torchia J, Yang W M, Brard G, Ngo S D, Davie J R, Seto E, Eisenman R N, Rose D W, Glass C K, Rosenfeld M G A complex containing N-CoR, mSin3 and histone deacetylase mediates transcriptional repression. Nature 1997 May 1; 387(6628):43-8
7—Nagy L, Kao H Y, Chakravarti D, Lin R J, Hassig C A, Ayer D E, Schreiber S L, Evans R M Nuclear receptor repression mediated by a complex containing SMRT, mSin3A, and histone deacetylase. Cell 1997 May 2; 89(3):373-80
8—Alland L, Muhle R, Hou H Jr, Potes J, Chin L, Schreiber-Agus N, DePinho R A Role for N-CoR and histone deacetylase in Sin3-mediated transcriptional repression. Nature 1997 May 1; 387(6628):49-55
9—Laherty C D, Yang W M, Sun J M, Davie J R, Seto E, Eisenman R N Histone deacetylases associated with the mSin3 corepressor mediate mad transcriptional repression. Cell 1997 May 2; 89(3):349-56
10—Zhang Y, Iratni R, Erdjument-Bromage H, Tempst P, Reinberg D Histone deacetylases and SAP18, a novel polypeptide, are components of a human Sin3 complex. Cell 1997 May 2; 89(3):357-64
11—Taunton J, Hassig C A, Schreiber S L A mammalian histone deacetylase related to the yeast transcriptional regulator Rpd3p. Science 1996 Apr. 19; 272(5260):408-11
12—Yang W M, Inouye C, Zeng Y, Bearss D, Seto E Transcriptional repression by YY1 is mediated by interaction with a mammalian homolog of the yeast global regulator RPD3. Proc Natl Acad Sci USA 1996 Nov. 12; 93(23):12845-50
13—Ayer D E, Lawrence Q A, Eisenman R N Mad-Max transcriptional repression is mediated by ternary complex formation with mammalian homologs of yeast repressor Sin3. Cell 1995 Mar. 10; 80(5):767-76
14—Schreiber-Agus N, Chin L, Chen K, Torres R, Rao G, Guida P, Skoultchi A I, DePinho R A An amino-terminal domain of Mxi1 mediates anti-Myc oncogenic activity and interacts with a homolog of the yeast transcriptional repressor SIN3. Cell 1995 Mar. 10; 80(5):777-86
15—Grunstein M Histone acetylation in chromatin structure and transcription. Nature 1997 Sep. 25; 389(6649):349-52
16—Wolffe A P Transcriptional control. Sinful repression. Nature 1997 May 1; 387(6628): 16-7
17—Mimori K, Druck T, Inoue H, Alder H, Berk L, Mori M, Huebner K, Croce C M Cancer-specific chromosome alterations in the constitutive fragile region FRA3B. Proc Natl Acad Sci USA 1999 Jun. 22; 96(13):7456-61
18—Menin C, Santacatterina M, Zambon A, Montagna M, Parenti A, Ruol A, D'Andrea EAnomalous transcripts and allelic deletions of the FHIT gene in human esophageal cancer. Cancer Genet Cytogenet 2000 May; 119(1):56-61
19—Voiculescu I, Back E, Schempp W. Homozygous condition for a BrdU-requiring fragile site on chromosome 12. Hum Genet. 1991 February; 86(4):416-7.
20—Sutherland G R Heritable fragile sites on human chromosomes: Factor affecting expression in lymphocyte culture. Am J Hum Genet. 31: 136-148.
21—Q Jiang, S Galiègue-Zouitina, C Roumier, M P Hildebrand, S Thomas, LJ Coignet. Genomic organization and refined mapping of the human silencing mediator of retinoid and thyroid hormone receptor (smrt) on chromosome 12q24.3. Cytogenet Cell Genet, 97: 217-220.

We claim:

1. A method of determining whether a human subject has an increased likelihood of cancer metastasis comprising the steps of:
a) providing a biological sample comprising nucleated cells from the subject;
b) culturing the cells;
c) adding to the cells of b) an agent known to induce breakage of chromosomal fragile sites; and
d) analyzing SMRT gene expression;
wherein a reduction in SMRT gene expression relative to a normal control is indicative that the individual has an increased likelihood of cancer metastasis, and wherein an absence of a reduction in SMRT expression is indicative that the individual does not have an increased likelihood of cancer metastasis.

2. The method of claim 1, wherein the reduction in SMRT gene expression comprises a reduction in SMRT mRNA.

3. The method of claim 2, wherein the reduction in SMRT mRNA comprises a transient reduction in SMRT mRNA, wherein the transient reduction occurs between approximately 2 and 5 hours after adding the agent known to induce breakage of chromosomal fragile sites to the cells.

4. The method of claim 2, wherein the SMRT mRNA is analyzed by RT-PCR.

5. The method of claim 4, wherein the RT-PCR is performed using a first primer comprising the sequence of SEQ ID NO:3 and a second primer comprising the sequence of SEQ ID NO:4.

6. The method of claim 2, wherein the SMRT mRNA encodes a protein having the sequence of SEQ ID NO:2.

7. The method of claim 2, wherein the SMRT mRNA comprises the sequence of SEQ ID NO:1.

8. The method of claim 1, wherein the reduction in SMRT gene expression comprises a reduction in SMRT protein.

9. The method of claim 8, wherein reduction in SMRT protein occurs approximately 18 hours after adding the agent known to induce breakage of chromosomal fragile sites to the cells.

10. The method of claim 1, wherein the human subject has been diagnosed with or is suspected of having a cancer selected from the group of cancers consisting of breast cancer, lung cancer, prostate cancer, melanoma, colorectal cancer, endometrial cancer and kidney cancer.

11. The method of claim 1, wherein the agent known to induce breakage of chromosomal fragile sites is a DNA polymerase inhibitor.

12. The method of claim 11, wherein the DNA polymerase inhibitor is aphidicolin.

13. The method of claim 1, wherein the biological sample comprises lymphocytes.

14. A kit for determining whether a human subject has an increased likelihood of cancer metastasis comprising: an agent known to induce breakage of chromosomal fragile sites, and at least one SMRT specific probe, wherein the probe is selected from the group of probes consisting of nucleic acid sequences comprising a sequence of sufficient complementary to SMRT mRNA to hybridize to SMRT mRNA; anti-SMRT antibodies or SMRT protein binding fragments thereof; and BAC clones selected from the group of BAC clones consisting of RP11-339B19, RP11-665C13, RP11-677L6, RP11-408118 and RP11-30G17, and combinations thereof.

15. The kit of claim 14, wherein the at least one probe is BAC clone RP11-665C13 or RP11-677L6.

16. A method for determining whether a human subject has an increased likelihood of metastasis comprising:
a) obtaining a biological sample from the individual, wherein the biological sample comprises nucleated cells;
b) performing fluorescent in situ hybridization (FISH) on the sample, wherein the FISH is performed using BAC clone RP11-665C13 labeled with a first fluorescent agent and BAC clone RP11-677L6 labeled with the first fluorescent agent, and a control probe labeled with a second fluorescent agent;
wherein co-localization of a fluorescent signal from the BAC clones labeled with the first fluorescent agent and the control probe labeled with the second fluorescent agent is indicative that the individual does not have an increased likelihood of metastasis,
and wherein a disrupted signal from the BAC clones labeled with the first fluorescent agent is indicative that the individual has an increased likelihood of metastasis.

17. The method of claim 16, wherein the human subject has been diagnosed with or is suspected of having a cancer selected from the group of cancers consisting of breast cancer, lung cancer, prostate cancer, melanoma, colorectal cancer, endometrial cancer and kidney cancer.

18. The method of claim 16, wherein the control probe is BAC clone RP11-469A24.

19. The method of claim 16, wherein the disrupted signal is a split signal.

20. A method for determining whether a human subject has an increased likelihood of cancer metastasis comprising the steps of:
a) providing a biological sample comprising nucleated cells from the subject; and
b) determining the presence or absence of a FRA12E fragile site;
wherein the presence of FRA12E fragile site is indicative that the individual has an increased likelihood of cancer metastasis, and wherein the absence of FRA12E is indicative that the individual does not have an increased likelihood of cancer metastasis.
